(19) United States
(12) Patent Application Publication
(10) Pub. No.: US 2025/0006072 A1
(43) Pub. Date: Jan. 2, 2025

(54) HEALTHCARE TRAINING AND CONTINUING HEALTHCARE EDUCATION SYSTEM AND METHODS

(71) Applicant: OOG, Inc., Las Vegas, CA (US)

(72) Inventors: Heather Hasson, Las Vegas, NV (US); Felipe Recalde, San Francisco, CA (US); Pamela Mehta, San Jose, CA (US); Sean Kenneth Clarke, Harlingen, TX (US)

(21) Appl. No.: 18/456,182

(22) Filed: Aug. 25, 2023

Related U.S. Application Data
(60) Provisional application No. 63/510,806, filed on Jun. 28, 2023.

(51) Int. Cl. G09B 5/02; G06N 20/00; G06Q 50/20; G16H 40/20

(52) U.S. Cl. CPC G09B 5/02; G06N 20/00; G06Q 50/2057; G16H 40/20; G06F 3/0482; G06F 40/20

(57) ABSTRACT

Methods, systems, and apparatus, including medium-encoded computer program products, including receiving, through a user interface, a request for a healthcare education content item including one or more healthcare education terms, providing, for presentation in the user interface, a subset of healthcare education content items, receiving a selection of a healthcare education content item of the subset of healthcare education content items, providing the selected healthcare education content item and the one or more reflective prompts for the selected healthcare education content item, receiving a user reflection responsive to a reflective prompt presented with the selected healthcare education content item, generating, by machine learned models, a user reflection score corresponding to reflection assessment criteria of the user reflection, classifying the user reflection based on whether the user reflection meets a threshold healthcare education credit criteria, and providing a notification including the classification of the user reflection.

### CROSS REFERENCE TO RELATED APPLICATIONS

This application claims the benefit to U.S. Patent Application 63/510,806, filed on Jun. 28, 2023, the contents of which are incorporated herein by reference in its entirety.

### TECHNICAL FIELD

This specification relates to healthcare training and continuing healthcare education.

### BACKGROUND

Continuing education (CE), for example, continuing medical education (CME) and continuing education unit (CEU), is a mandated requirement for healthcare providers to ensure they remain up-to-date with the latest research, clinical practices, and healthcare technologies throughout their careers. CE is implemented by licensing entities to promote learning, advance medicine, and establish certain standards of care among their boarded healthcare practitioners. Reflective learning is an approach to healthcare training and CE that emphasizes critical reflection on one's own experiences and the application of new knowledge and skills to one's clinical practice. This approach can encourage health care trainees and healthcare providers to think deeply about their practice, identify areas for improvement and development, and ultimately improve patient outcomes.

### SUMMARY

This specification describes technologies for an interactive platform for reflective learning-based healthcare training and continuing education.

In general, one innovative aspect of the subject matter described in this specification can be embodied in methods that include the actions of obtaining a plurality of healthcare education content items, each healthcare education content item comprising healthcare education content, generating, for each healthcare education content item of the plurality of healthcare education content items, contextual data including one or more reflective prompts for the content of the healthcare education content item, receiving, through a user interface, a request for a healthcare education content item, the request including one or more healthcare education terms, providing, for presentation in the user interface and responsive to the one or more healthcare education terms, a subset of healthcare education content items of the plurality of healthcare education content items, receiving, through the user interface, a selection of a healthcare education content item of the subset of healthcare education content items, providing, for presentation in the user interface, the selected healthcare education content item and the one or more reflective prompts for the selected healthcare education content item, receiving, through the user interface, a user reflection responsive to a reflective prompt of the one or more reflective prompts presented with the selected healthcare education content item of the plurality of content items, generating, by a plurality of machine learned models and from the user reflection and the contextual data, a user reflection score corresponding to a plurality of reflection assessment criteria of the user reflection, classifying the user reflection with the presented healthcare education content item based on whether the user reflection meets a threshold healthcare education credit criteria. The classifying includes, in response to determining that the user reflection score meets the threshold healthcare education credit criteria, classifying the user reflection as healthcare education credit qualified, and in response to determining that the user reflection score does not meet the threshold healthcare education credit criteria, classifying the user reflection as healthcare education credit unqualified, providing, for presentation in the user interface, a notification including the classification of the user reflection.

Other embodiments of this aspect include corresponding computer systems, apparatus, and computer programs recorded on one or more computer storage devices, each configured to perform the actions of the methods.

The subject matter described in this specification can be implemented in particular embodiments so as to realize one or more of the following technical advantages. Using reflective learning-based in healthcare education (e.g., continuing education and healthcare training) emphasizes learning as an active process that involves critically thinking about one's own experiences and reflecting on how to incorporate new information into one's practice. The interactive platform facilitates a user to achieve learning goals related to healthcare education at a user's own pace and in flexible locations and time frame, improving an efficiency in obtaining/maintaining healthcare education credits. By using targeted reflection prompts to the user's profile and learning objectives, the system can improve relevancy of the learning to the user and improve an effectiveness of the customized learning to achieving user-based learning goals, e.g., reducing a time the user must spend on the interactive platform to achieve their goals.

The platform tracks a user's CE requirements to assist a user in staying up to date on meeting the requirements for their professional licensing, removing the need for additional tracking/expenditure to maintain professional licenses. Using trained machine-learning models to provide the enriched HE content and review the user reflections can remove a requirement of a human reviewer to process and approve each item of HE content and the responsive user reflections, which can reduce a cost associated for the users to obtain CE credit and reduce a financial hurdle to maintaining a license.

In addition to using the interactive platform to maintain a required CE credits for licensing/boarding, a healthcare provider can integrate reflective learning as a regular practice that results in a constant, continual learning and development on topics that are directly relevant to the user's practice. For many healthcare providers and trainees, reflective learning can be a more effective approach for promoting lifelong learning, ongoing professional development, and improved patient outcomes.

In some implementations, the interactive platform can reduce a barrier to direct conversations between practitioners with overlapping interests and expertise, encouraging high-level conversations and development of new ideas. The platform allows for users to create and share healthcare-relevant content, which can propagate and promote new learning and techniques faster and to a wider audience than traditional forms of CE.

In some implementations, the interactive platform can track trending topics of conversation between users of the platform and provide feedback to affiliated licensing boards or certifying entities. Additionally, reviewing trends on the interactive platform can be used to refine and offer more focused content to users to reflect their educational or professional goals.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example operating environment of an interactive healthcare education system.

FIG. 2 is a flowchart of an example process for content creation using the interactive healthcare education system.

FIG. 3 is a flowchart of an example process for healthcare education content curation by the interactive healthcare education system.

FIG. 4 is a flowchart of an example process for generating reflective prompts by the interactive healthcare education system.

FIG. 5 is a flowchart of an example process of the interactive healthcare education system.

FIGS. 6A-6M depict various example views of a graphical user interface of the interactive healthcare education system.

FIGS. 7A-7F depict various example views of a graphical user interface of the interactive healthcare education system.

FIGS. 8A-8F depict various example views of a graphical user interface of the interactive healthcare education system.

FIG. 9 is a schematic diagram of a computer system.

Like reference numbers and designations in the various drawings indicate like elements.

### DETAILED DESCRIPTION

### Overview

The technology of this specification is directed towards an interactive platform for reflective learning-based healthcare education content for healthcare trainees and healthcare providers.

More particularly, these technologies involve an interactive platform for healthcare trainees and healthcare providers to earn and track reflective learning-based healthcare education credits (HEC) by performing reflective learning-based tasks in response to interacting with healthcare education content, e.g., videos, graphics, podcasts, live conferences, and textual-based healthcare education content. For example, the interactive platform can be used by healthcare providers to earn accredited continuing healthcare education (HE) credits required by boarding, credentialing and/or licensing entities (e.g., by state-based medical boards) by viewing healthcare education content and responding to reflective prompts selected based on the viewed healthcare education content. The interactive platform facilitates healthcare trainees and healthcare providers to generate interactive content, e.g., videos, graphics, etc., in a variety of fields and topics, which can be viewed by other healthcare trainees and healthcare providers for earning HE credits.

The interactive platform for healthcare education can be used by healthcare trainees and well as healthcare professionals to obtain new knowledge, develop expertise, enhance their practice, and stay current with up-to-date in medical developments. Additionally, the interactive platform can facilitate dialog and networking opportunities between healthcare professionals having overlapping expertise, field of practice, professional interests, or location-based commonalities. For example, the interactive platform can facilitate discussions of emerging techniques, topics of interest, professional networking, and career/training goals between healthcare professionals using the platform. The platform can facilitate forming intentional communities around expertise, professional goals, regions, and subject matter interests.

The interactive platform implements trained machine-learning models including generative artificial intelligence (e.g., large language models (LLMs)) to generate, enrich, and curate presentation of the reflective-based learning of healthcare education content. The LLMs can include, for example, decoder-only model(s), encoder-only model(s), or decoder-encoder model(s). The LLMs can be training using masked language modeling (MLM) objective. The LLMs can include deep learning transformer-based architecture. The LLMs can be optimized for their intended use cases, e.g., to classify input or generate output, that are specific to the healthcare education field, reflection-based learning, or the like. As such, the training data used to train and/or refine the LLMs can include, for example, healthcare-specific data, reflection learning-based data, etc. For example, training data can be generated specifically for training and/or refining the models described herein. Reinforcement learning can be used to refine the LLMs to improve their performance in the particular tasks described herein. In some instances, a human operator can provide feedback to one or more of the models to refine the model. The trained machine-learning models can also be used to process user-provided reflections in response to the healthcare education content and assess the quality of the user reflection-based learning from the viewed healthcare education content. The interactive platform can determine, from the scoring criteria generated by the trained machine-learning models, whether a healthcare education credit can be awarded in response to the user-provided reflection.

As used in this specification, a healthcare provider and healthcare trainee can include (but is not limited to), for example, a Nurse (RN, LVN, NP, etc.), Veterinarian, Doctor MD/DO, Dentist, Physician Assistant, Veterinary Technician, Naturopathic Doctor (ND), Pharmacist, Optometrist, Podiatrist, Medical Assistant, Social Worker, Athletic Trainer, Chiropractor, Dietician, Occupational Therapist, Physical Therapist, Respiratory Therapist, Radiology Technician, Phlebotomist, Dental Assistant (hygienist), Medical Laboratory Scientist, Medical Administrator (e.g., Biller, Medical Coder, etc.), Psychologist, Pharmacy Technician, Home Health Aide, Nursing Assistant, Diagnostic Medical Sonographer, Health Information Technician, Speech Language Therapist, Occupational therapy aid, Physical therapy aid, Surgical tech, Medical transcriptionist, Massage Therapist, Lab Animal Caretaker, Dispensing Optician, or another similar provider or trainee in a healthcare or patient care related field. A skilled artisan will appreciate that this is a non-exhaustive list of healthcare providers and healthcare trainees, and that a potentially larger scope of healthcare and/or patient care professions is possible.

As used in this specification, a healthcare education credit (HEC) can include, for example, healthcare education content that can be used towards directly or indirectly earning a certification, degree, license, boarding, or other form of documented education pathway for a healthcare provider or trainee. A healthcare education credit can be used by a healthcare provider or trainee to study for or as reference material for non-certification means, e.g., to study for an exam or for professional development. Examples of healthcare education credits can include, for example, continuing medical education (CME) credits, continuing education unit (HEU) credits, or another form of tracking HEC for the end goal of licensure or boarding.

### Example Operating Environment

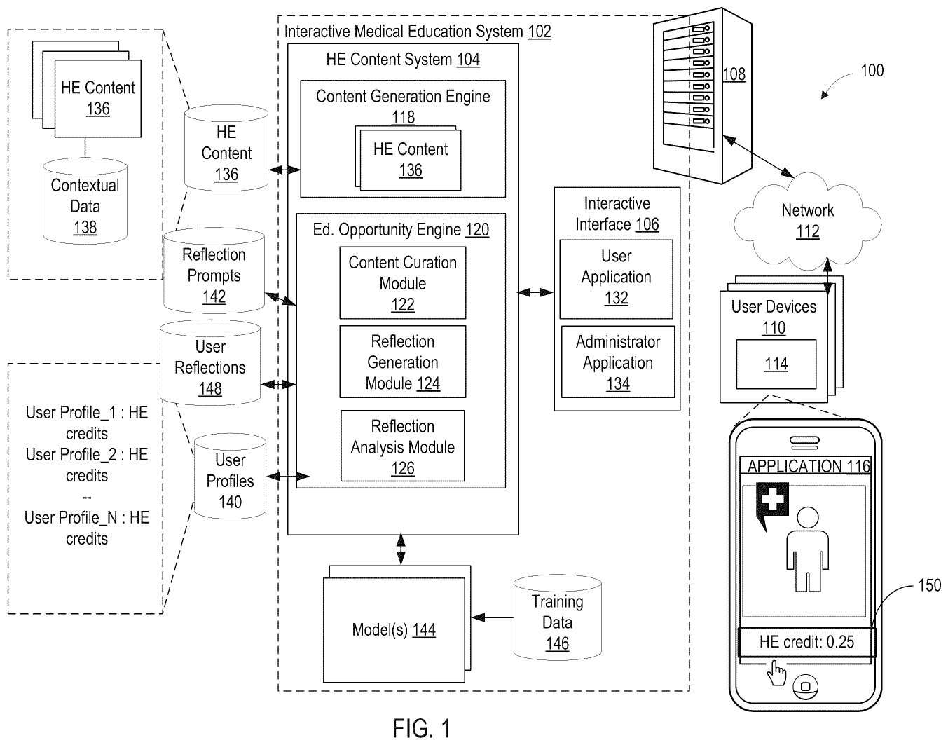

FIG. 1 is a block diagram of an example operating environment 100 of the interactive healthcare education system 102. Interactive healthcare education system 102 can be hosted on one or more local servers, a cloud-based service, or a combination thereof. System 102 includes a healthcare education (HE) content subsystem 104 and an interactive interface subsystem 106 and is hosted on one or more servers 108, e.g., cloud-based servers.

Interactive healthcare education system 102 can be in data communication with a network 112, where the network can be configured to enable exchange of electronic communication between devices connected to the network. The network may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. The network may include multiple networks or subnetworks, each of which may include, for example, a wired or wireless data pathway. The network may include a circuit-switched network, a packet-switched data network, or any other network able to carry electronic communications (e.g., data or voice communications). For example, the network may include networks based on the Internet protocol (IP), asynchronous transfer mode (ATM), the PSTN, packet-switched networks based on IP, X.25, or Frame Relay, or other comparable technologies and may support voice using, for example, VOIP, or other comparable protocols used for voice communications. The network may include one or more networks that include wireless data channels and wireless voice channels. The network may be a wireless network, a broadband network, or a combination of networks including a wireless network and a broadband network.

User devices 110 can include, for example, a mobile phone, tablet, computer, or another device including an operating system 114 and an application environment 116 through which a user can interact with the system 102. In one example, user device 110 is a mobile phone including application environment 116 configured to display healthcare education content. The user devices 110 can present, through application environment 116, an interactive healthcare education user interface 132, including healthcare education content. Details of the user interface 132 is described in further detail below.

Healthcare education (HE) content subsystem 104 includes a content generation engine 118, and an education opportunity engine 120. The education opportunity engine includes a content curation module 122, reflection generation module 124, and a reflection analysis module 126. Though described herein with reference to the content generation engine 118, and an education opportunity engine 120, content curation module 122, reflection generation module 124, and a reflection analysis module 126, the operations described herein can be performed by more or fewer sub-components.

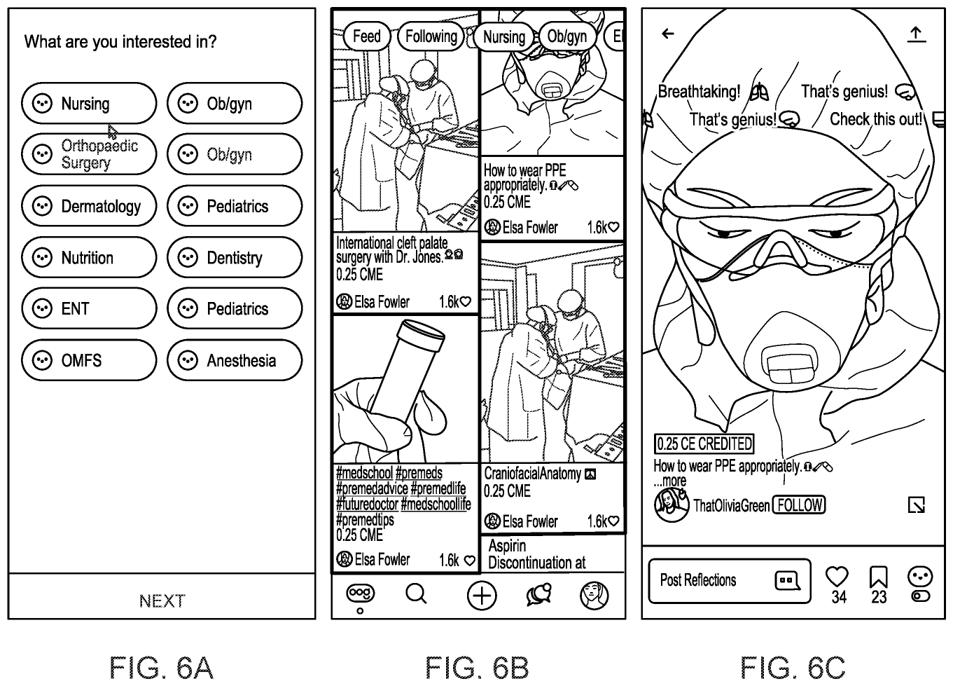

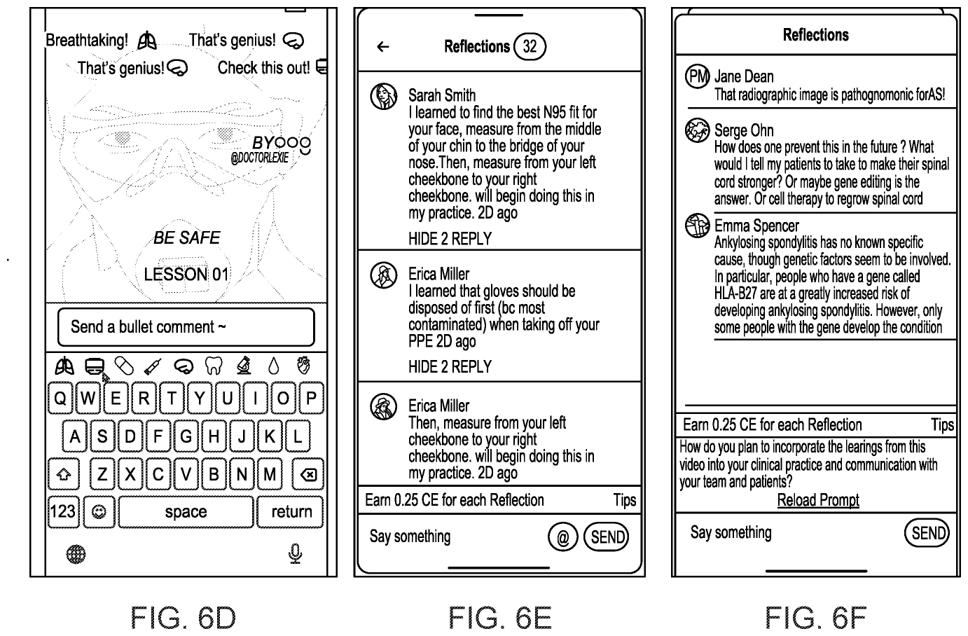

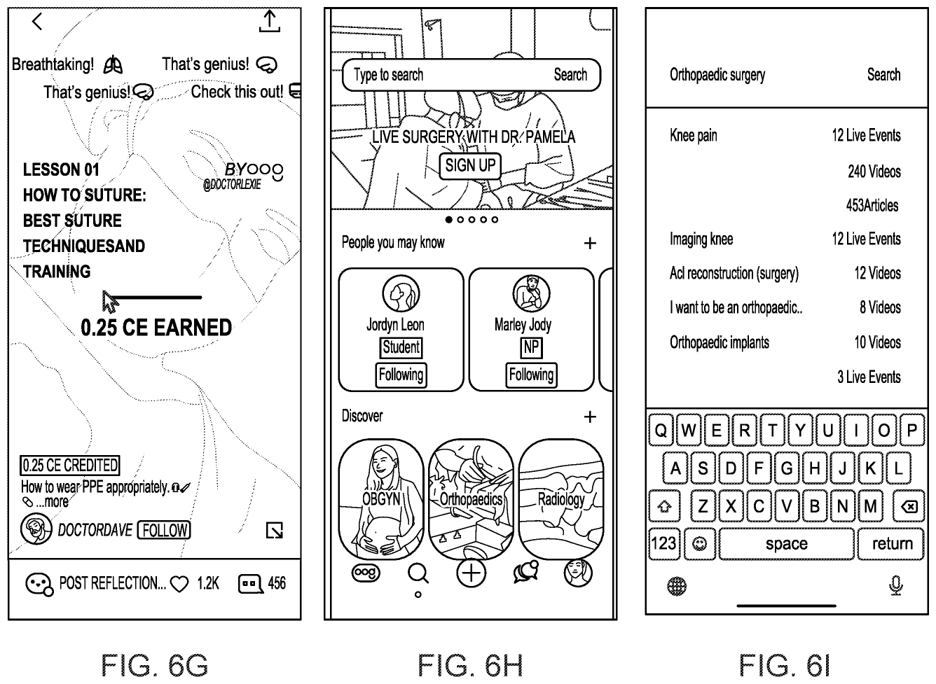

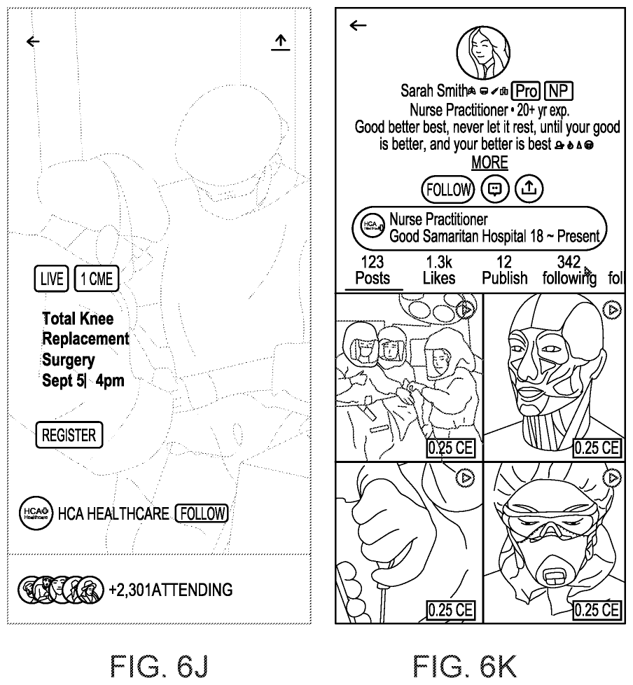

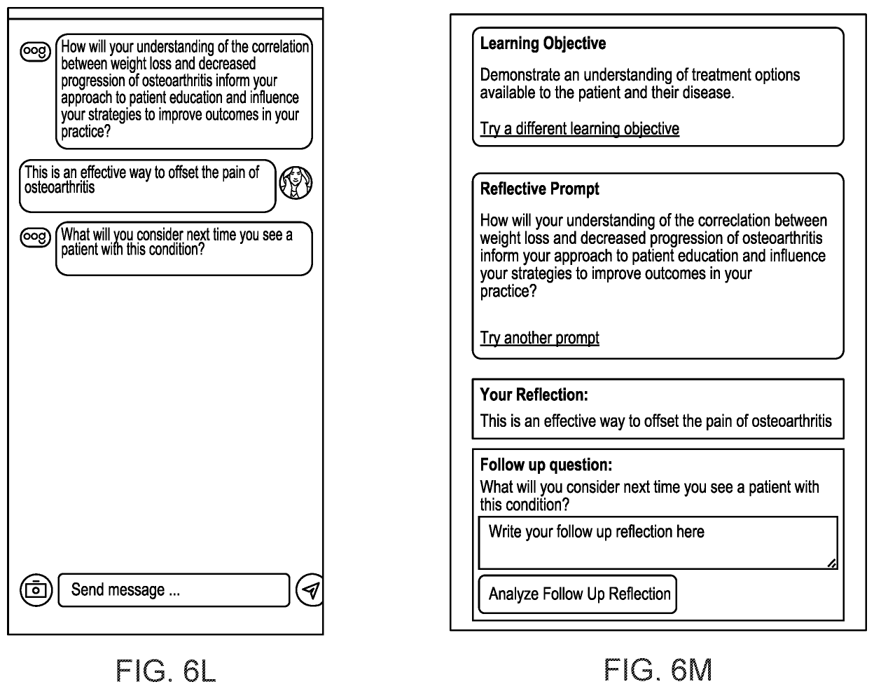

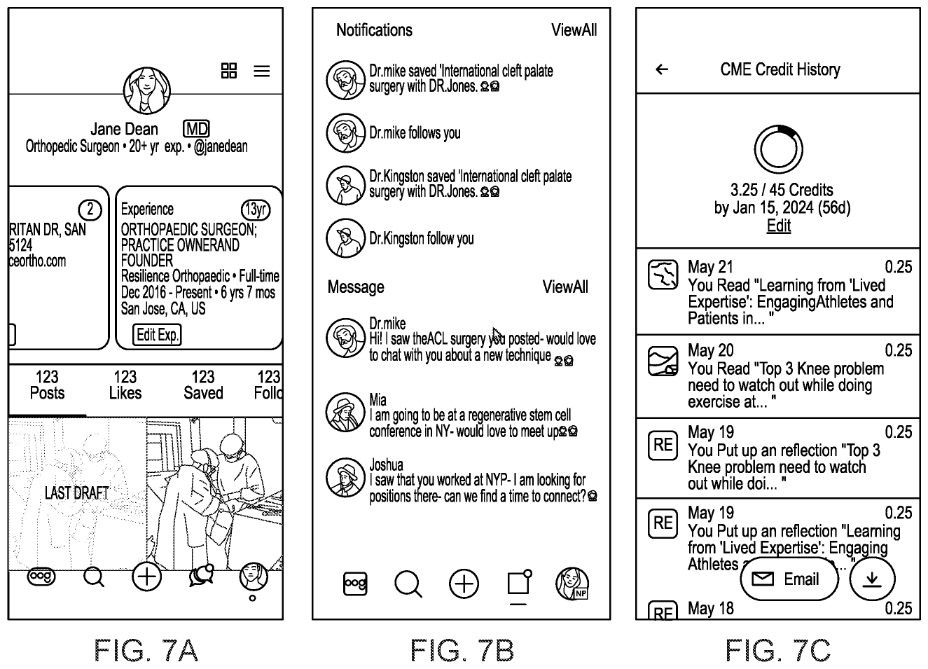

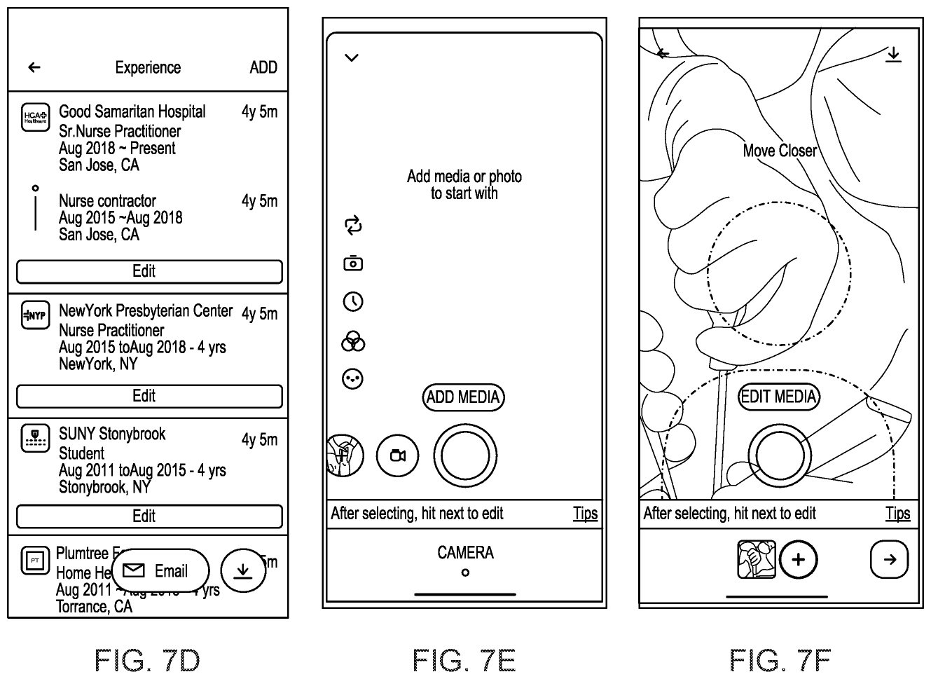

In some implementations, a user can be an end-user, for example, a healthcare professional, healthcare trainee, or the like. The user can interact with the system 102 through a user application interface 132 presented in an application environment 116 on the user device 110. For example, the user application interface 132 can be a graphical user interface (GUI) as depicted in the example user interface (UI) views of FIGS. 6A, 6B, 6C, 6D, 6E, 6F, 6G, 6H, 6I, and 6K and FIGS. 7A, 7B, 7C, 7D, 7E, and 7F, including functionality, e.g., for viewing and interacting with HE content, providing reflections responsive to the HE content, and earning healthcare education credits (HEC). Further details related to the user interface are described below.

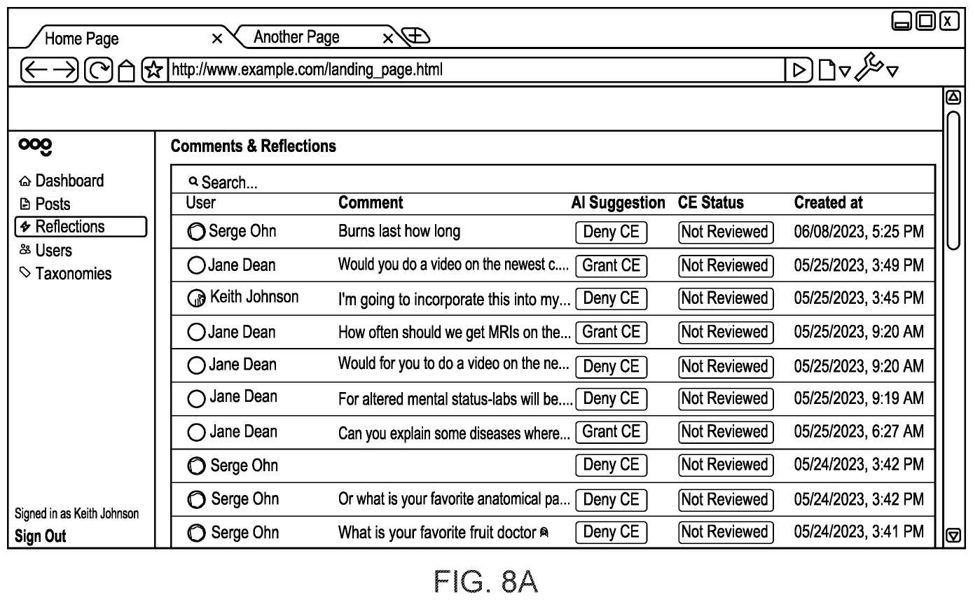

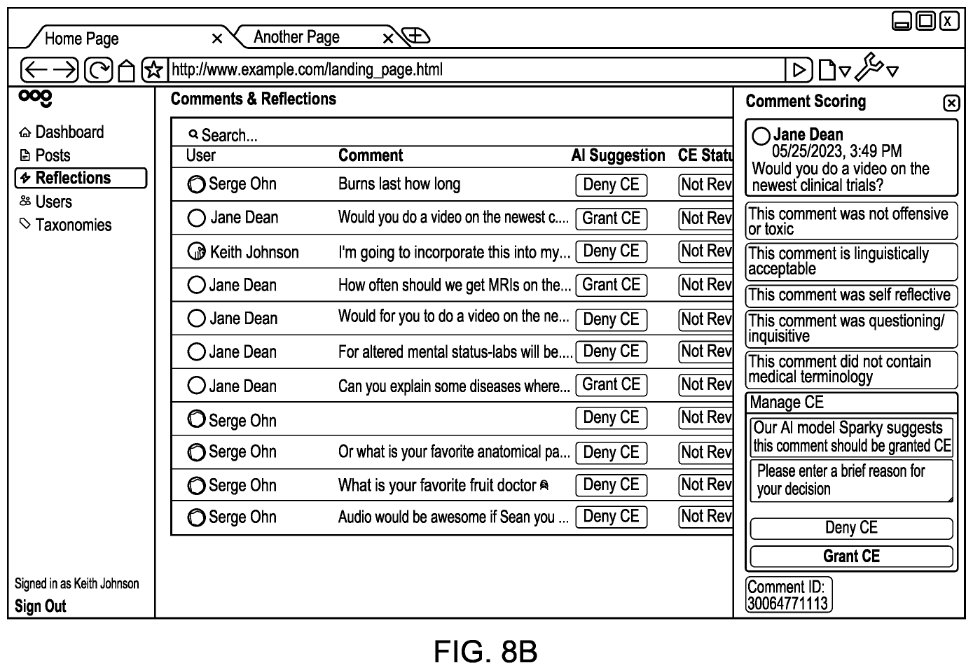

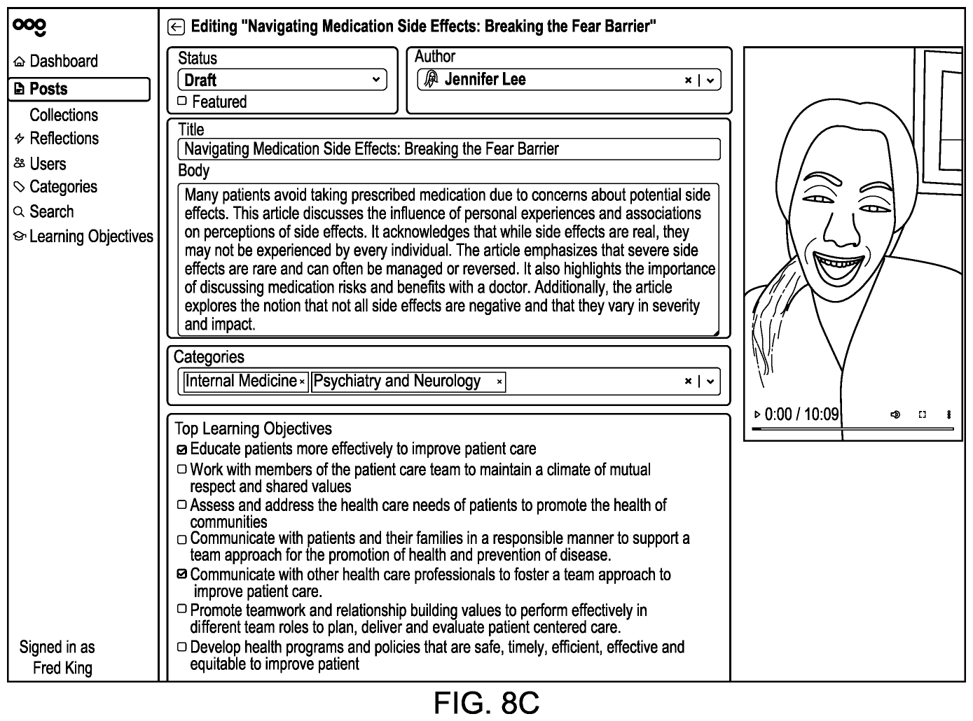

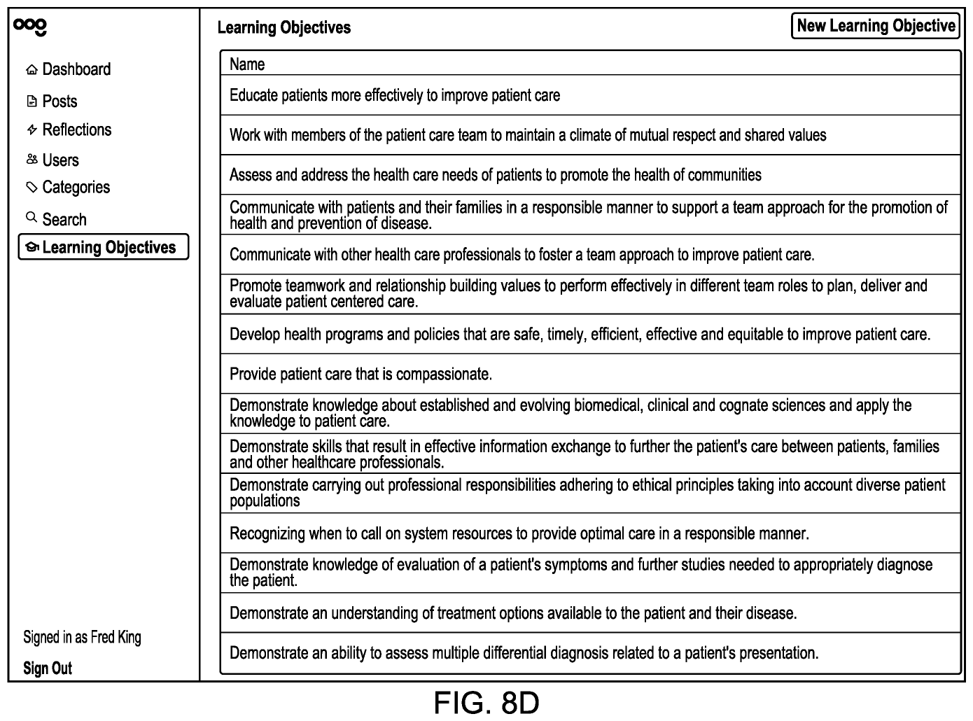

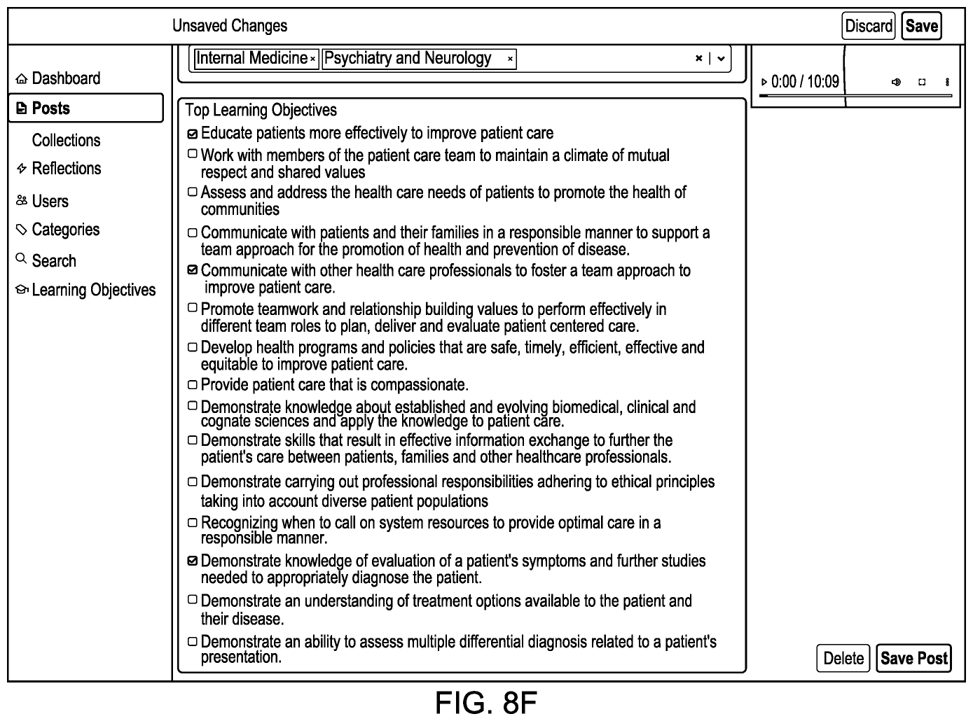

In some implementations, a user can be an administrative or super user, for example, a healthcare educator, an employee of a licensing entity (e.g., medical boards, nursing boards, etc.), or the like. The user can interact with the system 102 through an administrator interface 134 presented on the user device 110. For example, the administrator interface 134 can be a graphical user interface as depicted in the example UI views of FIGS. 8A, 8B, 8C, 8D, 8E, and 8F, including functionality, e.g., for viewing and interacting with content, managing content, HE credits, and reflection assessment criteria, etc. Further details related to the administrator interface are described below. The user application interface 132 and administrator interface 134 can include different functionality and can grant access to different levels of the healthcare education content subsystem 104.

Content generation engine 118 is configured to obtain HE content 136 as input, e.g., facilitate generation and/or submission of media content related to healthcare education to the system 102. For example, HE content can include videos, informational graphics, audio, text, etc. that include healthcare education content. Healthcare education content can relate to one or more various healthcare subtopics. For example, healthcare content can be best practices related, e.g., taking a patient history, wearing personal protective equipment, etc. In another example, healthcare education content can relate to different medical fields, e.g., pulmonology, cardiology, dermatology, veterinary medicine. In another example, healthcare education content can relate to emerging techniques, e.g., a new suturing technique, etc. In another example, healthcare content can be related to other healthcare topics, e.g., medical ethics, patient advocacy, public health, etc. The HE content 136 can be provided to the content generation engine 118 by a user through user interface 132 presented the application environment 116 on a user device 110.

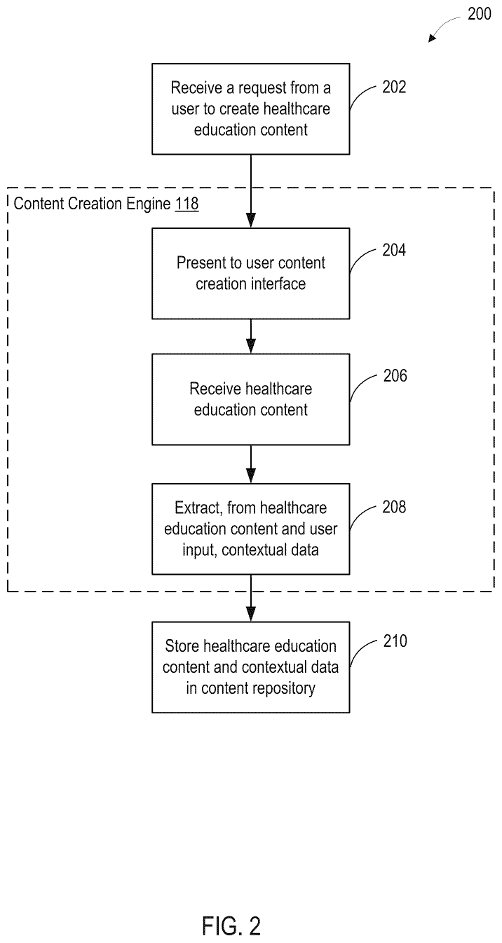

In some implementations, content generation engine 118 can receive a request as input to generate new healthcare education content from a user through the user interface 132 in the application environment 116. The content generation engine can, through user interface 132, guide the user through an HE content generation process, for example, as described in further detail with reference to FIG. 2.

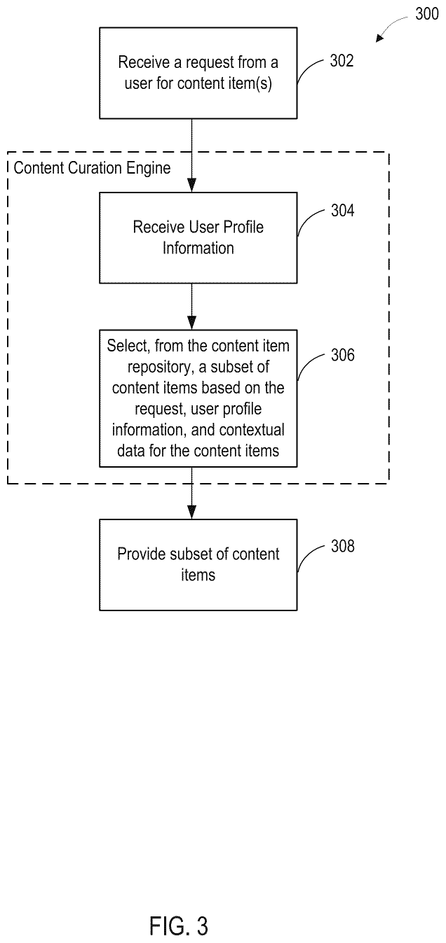

In any case, the content generation engine 118 can obtain the HE content 136 and process the HE content to generate enriched HE content as output, including generating contextual data 138 for the HE content 136. Contextual data 138 includes for example, learning objectives, main discussion points, titles, keywords, reflective prompts, expertise level of the user, tags/labels with applicable medical specialties, etc. Contextual data can be generated by the system 102. In some implementations, contextual data can be additionally provided by a user (e.g., content creator or administrator of system 102) for the HE content. For example, an administrator can generate the contextual data using an administrator interface, e.g., as described in further detail with reference to FIGS. 8C-8F below. Generation of the contextual data is described in further detail with reference to FIG. 3.

In some implementations, the system 102 generates contextual data by extracting, from the HE content, a transcript of the HE content (e.g., a transcript of a video, audio, or graphical display). The system 102 then provides the transcript of the HE content to a trained machine-learning model 144 (e.g., a large language model (LLM)) to extract, from the transcript, a list of main discussion points. A list of learning objectives can be generated from the main discussion points (e.g., using the LLM, by a human operator, or a combination thereof). Additionally, the system 102 can generate keywords, a title, and other contextual information from the transcript using a combination of a model 144, a human operator, or both. In some implementations, a human operator (e.g., a content creator or an education administrator) can tag/label the HE content to provide additional context to the system 102 when generating the contextual data. For example, a content creator can provide suggested keywords, technical areas, summary points, etc., representative of the HE content for use by the system 102.

In some implementations, the trained machine-learning model 144 can be trained and/or refined using training data generated from associations between discussion points and learning objectives, e.g., labeled by a human operator. For example, training vectors can be generated from previously enriched HE content where a human operator has identified main discussion points and learning objectives from the transcript of the HE content.

A repository of the enriched HE content 136 can be generated covering a range of topics, educational goals, expertise levels, etc. The repository can be indexed and classified, e.g., using SQL, Vector database, or another database management system, to assist in making the repository of videos searchable. For example, a Vector database can be used to improve a text (e.g., context) used to create the vectors and improve the training vectors provided to the model(s) 144.

System 102 includes a repository of user profiles 140. User profiles can be generated based on user provided feedback through the user application interface 132, e.g., as depicted in the UI views shown in FIGS. 7A-7F. In some implementations, user profiles 140 can include data collected by the system 102 through interactions of the user with the user interface. For example, a user can select to opt-in to allow the system 102 to generate curated HE content based in part on a user's past interactions with the HE content 136. User profile 140 can include for example, a user's healthcare education, field(s) of expertise, learning goals, professional networks, healthcare/medical interests, etc., e.g., as depicted in example UI view of FIG. 7A. System 102 can provide, to the user in a notifications view of the user profile, one or more notifications related to the user's interactions with the system 102, e.g., as depicted in example UI view of FIG. 7B. A user can additionally provide professional/educational background, A user profile 140 can include a shareable resume accessible to other users of the system 102, e.g., as depicted in example UI view of FIG. 7D. At times, system 102 can access the user profile 140 to customize one or more items of the user application interface 132, e.g., content curation, in response to user profile 140. Further details of the curation process by the system 102 are described below with reference to FIG. 3.

Education opportunity engine 120 receives, from a user through the user interface 132 presented in the application environment 116 on user device 110, a request for HE content 136. A request for HE content 136 can include, for example, a search query entered by the user, a keyword selection by the user, etc. In some implementations, a request for content by the user can be the user opening the user application interface 132 or selecting a landing page of the interface 132. A search query can be entered by a user through the user interface as a text-based query, or voice-input query. The user can optionally select from a set of suggested queries, e.g., related to trending topics, previous searches entered by the user, or related searches.

Content curation module 122 can receive the request for HE content as input and provide, from the repository of HE content 136, a selection of HE content responsive to the request as output. Further details of the operations of the content curation module are described with reference to FIG. 3.

The system 102 selected HE content is presented to the user in the interface 132 of the application environment 116, e.g., as depicted in UI view of FIG. 6B. The user can review the selection of curated HE content and elect a HE content item of interest or elect to update the request for HE content, e.g., if none of the presented HE content options are satisfactory. Reflection generation module 124 of the education opportunity engine 120 can receive a selection of an HE content of the presented HE content in the interface 132 as input. The reflection generation module 124 can use one or more trained machine-learning models 144 to generate, from the contextual data 138, the user profiles 140, or a combination thereof, one or more reflective prompts 142 for the HE content 136.

Models 144 can include, for example, large language models (LLMs) and/or multimodal large language models (MLLMs), where the models 144 are trained using training data 146. For example, an LLM to generate reflective prompts 142 can be trained using a repository of healthcare/medical terminology, reflective learning-based terminology, and reflective-learning based phrases. The LLM-type models 144 can receive a textual-based input extracted from the contextual data, user profile data, reflective learning-based terminology and phrases, etc., and generate the one or more reflective prompts 142 as output. Further details of training the models 144 is described below.

In some implementations, the LLM-type models 144 can be fine-tuned pretrained language models by supervised training to complete a specific task, e.g., generating reflective prompts 142. At times, reinforcement learning can be used to refine the model(s) 144 using feedback from a human operator, e.g., a user of administrator interface 134 as depicted in the example UI view of FIG. 8C. For example, a user of the administrator interface 134 can review reflective prompts generated by the model 144 and provide updated training data 146 to refine/adjust the performance of the model. Further details are described below.

In some implementations, reflection generation module 124 can generate one or more reflective prompts 142, e.g., targeted reflective prompts, based in part on the contextual data 138 for selected HE content and user information stored in user profile 140. For example, the one or more targeted reflective prompts 142 can be customized for a user profile, e.g., based on a user's history, medical/professional interests, expertise, learning objectives (e.g., a user's stated learning goals), etc.

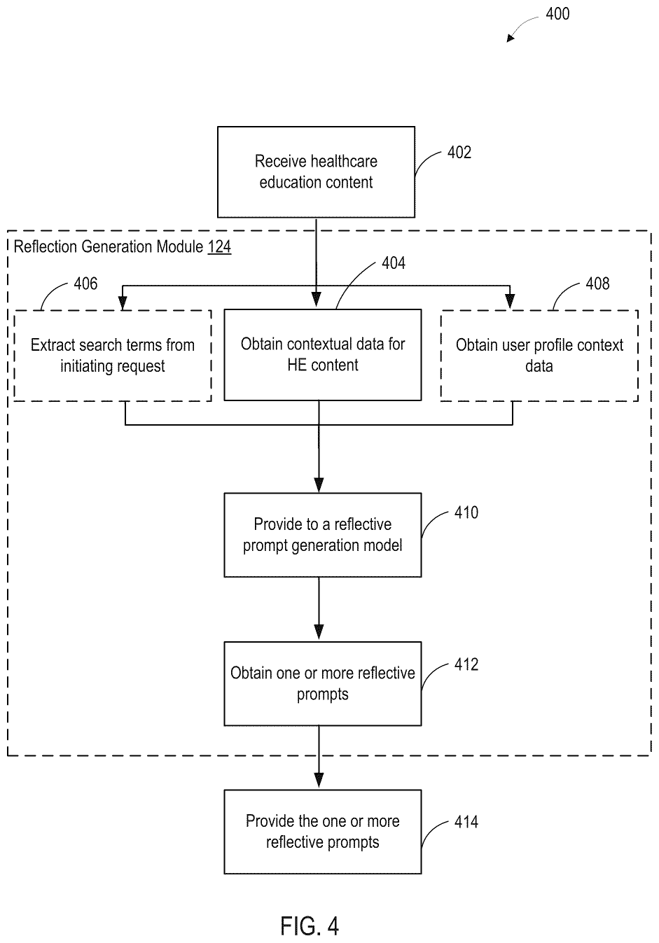

In some implementations, the reflection generation module 124 can generate one or more reflective prompts 142 for the HE content 136, e.g., generalized reflective prompts, based on the contextual data 138 that are non-specific to the particular user profile of the user that selects the HE content. For example, each user can receive the same set of one or more generalized reflective prompts. Further details related to the operations of the reflection generation module 124 are described with reference to FIG. 4.

Education opportunity engine 120 provides, for presentation through the user interface 132 on the user device 110, the one or more reflective prompts. In some implementations, one reflective prompt is presented at a time, e.g., in an overlay or pop-up window in the user interface, where a user can select to respond to the reflective prompt. Alternatively, the user can elect to request to view a different reflective prompt of the one or more prompts. The user can elect to respond to one or more reflective prompts in response to the HE content and submit the reflection through the user interface 132, e.g., as depicted in the example UI view of FIG. 6F.

Reflection analysis module 126 receives, as input, the user submitted reflection in response to the reflective prompt. Additionally, reflection analysis module 126 receives the reflective prompt to which the user responded, the HE content contextual data 138 and (e.g., optionally) user profile 140 as input. The user reflection 148 includes a text-based reflection, i.e., candidate text. Additionally, or alternatively, the user reflection can include additional formats of the reflective content. For example, a user reflection can include graphical format, e.g., meme, emoji, etc. In another example, a user reflection can be audio based, e.g., a recorded reflection of the user's voice. In another example, a user reflection can be video-based, e.g., a recorded video of the user providing their reflection.

In any case, the user reflection 148 is processed, e.g., using natural language processing, speech recognition, speech-to-text, etc., to extract candidate text representative of the user reflection. Candidate text can be, for example, a full textual transcript of the user reflection. In another example, candidate text can be a parsed format of the user-input reflection, e.g., a set of keywords and phrases. In some implementations, a convolutional neural network (CNN) may be used to process the user reflection to generate a candidate text including a set of keywords and phrases. The candidate text of the user reflection 148 is provided, by the reflection analysis module 126, to multiple trained machine-learning models 144 to generate a set of scoring criteria for the user reflection.

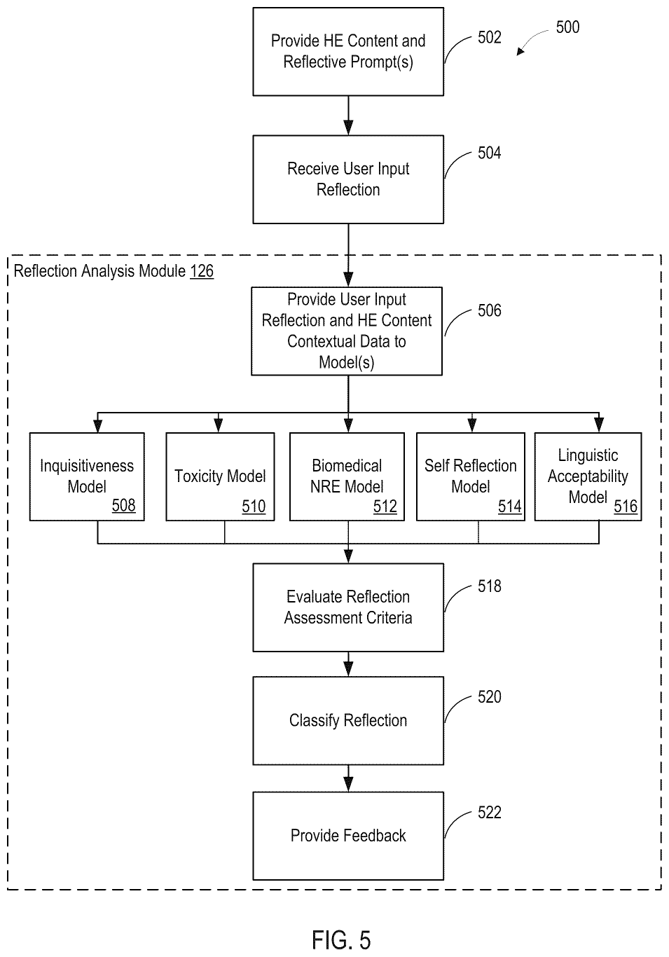

In some implementations, as described in further detail below with reference to FIG. 5, the multiple trained machine-learning models 144 include (A) an inquisitiveness model, (B) a toxicity model, (C) a biomedical NRE model, (D) a self-reflection model, and (D) a linguistic acceptability model. The operations of each of the aforementioned models are discussed in further detail with reference to FIG. 5. Each of the multiple models 144 are configured to receive, as input, the candidate text of the user reflection, and provide a scoring value for a reflection assessment criterion as output. The scoring criteria can be, for example, an output value between 0 and 1. Reflection analysis module 126 can evaluate, from the output scoring values of the multiple models 144 in response to the user reflection 148, whether the user reflection meets threshold reflection assessment criteria to receive a corresponding HE credit.

In some implementations, a user of the administrator interface 134 can adjust the threshold criteria for one or more of the outputs of the models 144 for the reflection analysis module 126 to determine whether to award an HEC. For example, a user may interact with the administrator interface, e.g., as depicted in UI views of FIGS. 8A-8B, to adjust a threshold criteria for one or more outputs of the models 144.

In some implementations, the reflection analysis module 126 includes a weighted scoring criteria, where an output of one or more of the models 144 can be weighted with respect to at least one other output of the models 144. A user of the administrator interface 134 can adjust, through the interface 134, a respective weighting of the output of the one or more models 144. For example, the output value of the inquisitiveness model can be weighted lower with respect to an output value of the biomedical terminology model. Further details are described with reference to FIG. 5.

The education opportunity engine 120 can provide, for presentation in user interface 132 in an application environment 116 of the user device 110, a notification 150 including whether or not the system 102 has granted the user an HEC for completing the HE content and user reflection. For example, in the instance where the user has earned the HEC, system 102 updates user profile 140 to include the earned HEC (e.g., updates a total HEC earned by the user).

In some implementations, when system 102 determines that the user's reflection does not meet a threshold criteria for earning the HEC, the system 102 provides feedback to the user. For example, the feedback can include missing criteria (e.g., “Your reflection did not include sufficient medical terminology” or “Your provided reflection was flagged as including toxic language.”). In another example, the feedback can provide suggested improvements to assist that user in providing qualifying reflections in response to future healthcare education opportunities.

In some implementations, in instances in which the system 102 does not classify the user reflection as earning an HEC (i.e., the system determines that the user's initial reflection does not meet a threshold criteria for earning the HEC), the system 102 generates a follow-up reflective prompt (e.g., a “nudge”) to prompt the user to provide a second user reflection. The follow-up reflective prompt can include keywords, phrases, and/or cues to elicit additional reflection-based learning from the user. At times, additional user reflective prompts can be provided to the user, e.g., in a chat-format environment, to prompt the user to provide multiple reflection-based learning responses to the HE content. The follow-up reflective prompts can assist the user in earning the HEC credit by engaging with the user to perform the qualifying reflection-based learning responsive to the HE content. In this way, the follow-up reflective prompt can increase an effectiveness of the HE content interaction and increase a conversion of a user's interactions with the system 102 into meaningful, reflection-based learning and earned HEC.

The system 102 can process the initial user reflection and the secondary user reflection(s) to classify the combined user reflections as earning an HEC or not earning an HEC from a cumulative score corresponding to all the user reflections submitted by the user responsive to the HE content. Each of the multiple models 144 can receive candidate text of the two (or more) user reflections and provide scoring values for the respective reflection assessment criterion as output. A cumulative score is generated from the combined user reflections by combining (e.g., adding) the scoring values of the user reflections output by the models 144 for each of the reflection assessment criteria. The reflection analysis module 126 can evaluate, from the output scoring values of the multiple models 144 in response to the combined user reflections, whether the combined user reflections meets the threshold reflection assessment criteria to receive a corresponding HE credit.

In some implementations, system 102 can provide the scoring criteria for the first user reflective prompt to the reflection prompt generator to generate a follow-up reflective prompt using the scoring criteria that is determined to not meet the threshold criteria. In other words, a scoring value for a reflection assessment criterion output by one or more of the models 144 for the initial user reflection that fails to meet a threshold criterion for earning the HEC can be used as input to the reflection prompt generator to produce a follow-up reflective prompt. For example, if the user's initial reflection is determined to not meet a threshold scoring value for the medical terminology reflection assessment criterion, the reflection prompt generator can generate a secondary reflection prompt to request additional medical-based terminology from the user. In another example, if the user's initial reflection is determined to not meet a threshold scoring value for linguistic acceptability (e.g., the user's reflection includes fewer than a threshold number of words), the reflection prompt generator can generate a secondary reflection prompt to elicit additional user reflection in response to HE content.

In some instances, the follow-up reflective prompt(s) are selected by system 102 from a repository of follow-up reflective prompts. The follow-up reflective prompts can be selected by the reflection prompt generator from multiple categories of reflective prompts from the repository of reflective prompts related to the different reflection assessment criterion. In other words, the secondary reflection prompt is context sensitive to the initial reflective prompt and the scoring values generated for the initial user reflection.

In some instances, secondary reflection prompts can be generated responsive to two or more scoring criteria not meeting a threshold criteria. The system 102 can select, from the determination that the initial user reflection does not meet one or more threshold scoring criteria, a follow-up reflective prompt and provide the follow-up reflective prompt to the user in the UI of the application environment. For example, if an initial user reflection does not meet a threshold scoring criteria for medical terminology and linguistic acceptability, the follow-up reflective prompt is selected to cue the user to provide a secondary user reflection including additional medical terminology and sentence complexity/length.

At times, as depicted in the example UI view of FIG. 6L, the reflective prompts (including the initial and the secondary reflection prompts) are presented to the user in a conversational style, e.g., in a chat format. The chat-style layout for the multiple reflective prompts and user reflections responsive to the prompts can be nested, e.g., as nested comments, or presented as a back-and-forth chat format. As depicted in the example UI view of FIG. 6M, the user may view a learning objective for the selected HE content and can select to view an analysis of each submitted user reflection for whether the cumulative scoring values meet a threshold for earning an HEC. Additionally, the user can select to respond to a different reflective prompt.

In some implementations, the system 102 can process the conversion success of reflective prompts into earned HEC and use the feedback to update the reflective prompts used in the initial reflective prompts and/or the follow-up reflective prompts for the HE content. For example, if the system 102 determines that a reflective prompt results in user reflections that do not qualify for an earned HEC at least a threshold percentage of the instances in which it is provided, the system 102 (or an administrator of system 102) can discard or edit the reflective prompt.

In some implementations, as described with reference to UI views depicted in FIGS. 8A-8C, feedback in response to a user's reflection is viewable by an administrator through the administrator interface 134.

In some implementations, as described with reference to FIG. 1, the interactive healthcare education system 102 facilitates content creation of healthcare education (HE) content. Content creators of the HE content can be, for example, other users of the interactive platform, medical educators, members of licensing entities, or other interested parties. FIG. 2 is a flowchart of an example process 200 for content creation using the interactive healthcare education system. For convenience, the process 200 will be described as being performed by a system of one or more computers, located in one or more locations, and programmed appropriately in accordance with this specification. For example, an interactive healthcare education system, e.g., the system 102 of FIG. 1, appropriately programmed, can perform the process 200.

The system 102 receives a request from a user to create content 202. The system 102 provides, for presentation through a user interface on a user device, a user content creation interface 204. For example, as depicted in an example UI views of FIG. 7E and 7F, a user can select, in the user interface 132, to create an item of content using a camera of the user device 110 (e.g., by selecting “shoot”), using a template (e.g., by selecting “template”), or from album repository of stock photos, videos, graphical representations, etc. (e.g., by selecting “album”).

In some implementations, system 102 includes content creation options for live-based HE content, e.g., webinars or live-streamed classes. Content creators can generate course-listings for live-based HE content through the system 102 (e.g., using user interface 132 or administrator interface 134). System 102 can be configured to host and broadcast live-based HE content, where a content creator may livestream HE content and one or more other users of the system 102 can access to view and interact with the live-based content.

The system obtains, through the user interface, healthcare education content 206. The system 102 can receive the HE content input through the user interface, e.g., as a content upload to the system 102, or as HE content created through a content creation functionality of the content generation engine 118. The system 102 can receive the HE content in various formats, e.g., video format, text-based formats, audio and/or visual formats, etc. The system 102 can limit HE content provided by a user to particular formats (e.g., mp4, jpeg, rtf, etc.) or can optionally include a conversion functionality to convert HE content to usable formats by the system 102. The system can additionally receive, for the healthcare education content, user-provided information (e.g., taxonomies) related to the HE content. For example, tags/labels, keywords, title, or other descriptive information related to the learning objectives, areas of expertise, or subject matter included in the HE content.

The system extracts, from the healthcare education content and user provided input, contextual data for the healthcare education content 208. Extracting contextual data can include generating a transcript of the HE content, e.g., using speech to text recognition and natural language processing. The system can then extract, by one or more models 144 and from the transcription of the HE content, a set of key discussion points from the transcription. The system can generate, from the set of key discussion points, relevant learning objectives. Discussion points are high-level overview summary statements of the content of the HE content item. For example, discussion points can include “new suturing technique for closing face wounds” or “proper way to put on N95 masks to minimize contamination risk.” Relevant learning objectives (e.g., also referred to herein as “learning goals”) are the objectives of a user when interacting with the HE content. Learning objectives can include, for example, filling gaps in knowledge, skills, and attitudes the user is trying to address, integrating new learning with existing knowledge and past experiences to incorporate into current or future practice, or identifying practice needs or learning.

In some implementations, system 102 can present in a user interface one or more of the key discussion points and relevant learning objectives extracted from the HE content for review by a user (e.g., a content creator or administrator of the system 102). For example, a user may select from a list of relevant learning objectives suggested by the system 102 for the HE content, where the selected learning objectives are stored with contextual data of the HE content, e.g., as described in further detail with reference to FIGS. 8C-8F below.

In some implementations, extracting contextual data includes receiving, from a user (e.g., content creator) through a user interface, one or more relevant learning objectives for the HE content. For example, the system can provide a list of suggested learning objectives (e.g., extracted from the discussion points or a standard set of learning objectives) and the user may select from the provided list and/or enter their own learning objectives for the HE content.

In some implementations, the system generates reflective prompts responsive to the HE content and the contextual data of the HE content. As discussed above, the reflective prompts can be generalized to the HE content and not specific to a particular user (e.g., without considering the user information of the user profile). For example, the reflective prompts can be directed towards learning objectives (e.g., increasing expertise in a topic, broadening practice, refining a technique, learning new developments in a medical field, etc.).

The system stores the healthcare education content and contextual data in a healthcare education content repository 210. The repository of healthcare education content 136 can be indexed and searchable, e.g., using a database management system, such that items of HE content can be accessible responsive to a user request for content.

In some implementations, as described with reference to FIG. 1, the interactive healthcare education system 102 facilitates content curation of healthcare education content. FIG. 3 is a flowchart of an example process 300 for healthcare education content curation by the interactive healthcare education system. For convenience, the process 300 will be described as being performed by a system of one or more computers, located in one or more locations, and programmed appropriately in accordance with this specification. For example, an interactive healthcare education system, e.g., the system 102 of FIG. 1, appropriately programmed, can perform the process 300.

The system receives a request from a user for healthcare education content item(s) 302. A request for HE content 136 can include, for example, a search query entered by the user, a keyword selection entered by the user, etc. The user can optionally select from a set of suggested queries (e.g., trending topics) presented in the user interface 132, e.g., as depicted in example UI views of FIGS. 6H and 6I. The request can include one or more search terms which the system 102 may parse into a format (e.g., keywords and/or phrases) compatible with a search of the HE content repository, e.g., a SQL search, Vector-based search, or similar.

The system receives user profile information for the user 304. The system 102 can receive user profile information including a user's learning objectives, past search and view history, level/area of expertise, etc. For example, the system 102 can use the user profile information to select HE content items that are compatible with a user's level of expertise in the field of the HE content (e.g., providing high-level technique content to a 20-year experienced professional in the field or providing an introductory level content to a healthcare student).

The system selects, from the healthcare content repository, a subset of content items based on the request, user profile information, and contextual data for the content items 306. The system 102 can select a subset of HE content items (e.g., one or more), where each selected HE content item in the selected subset has contextual data meeting at least a threshold relevancy to the search criteria. Search criteria includes, for example, the search query terms, user profile information, etc. In some implementations, the threshold relevancy can be determined by an overlap (e.g., threshold matching) of vectors generated for each of the request and the HE content items.

In some implementations, the system can customize the curated content based in part on historical user behavior, e.g., on conversation topics for user, video interactions, time spent interacting with content. The system 102 can select HE content not previously viewed by the user, e.g., to prevent the user from rewatching content. For example, for a user spending a threshold amount of time looking at HE content related to elbows, the system 102 may select to provide the user with curated content including HE content related to elbows or generally related to hand and forearm orthopedics.

In some implementations, the system 102 generates a customized (e.g., curated) feed including a categories breakdown, where a user can filter down by topic or practice. For example, the system 102 can present topics for filtering the curated content, e.g., as depicted in an example UI view of FIG. 6B.

In some implementations, system 102 includes a subscription-based curation for particular categories, content creators, or the like. For example, a user can opt-into following content for a particular content creator, e.g., as depicted in an example UI view of FIG. 6K, where the system 102 will preferentially include content from content creators whom the user has elected to follow. Additionally, a user can elect, through the user interface, to view content filtered by content creator, e.g., as depicted in the example UI view of FIG. 6K.

In some implementations, system 102 provides, for presentation in the user interface controls to filter the HE content by time, media type, length of expected interaction, etc. For example, the system 102 can filter the HE content based on a length of the expected interaction (e.g., a length of the video, an estimated reading time for text-based content). A user looking to earn an HE credit but with limited time to do so can elect to filter by length of expected interaction to find a suitable HE content item fitting their time limitations, which can increase an efficiency of the HE credit process for a user.

The system provides, for presentation in a user interface presented on a user device, the subset of HE content items 308. The system 102 can provide, for presentation in the user interface 132, the subset of HE content items, e.g., as depicted in the example UI view of FIG. 6B. In some implementations, the system 102 can provide data including search history and returned HE content items for review by an administrator in the administrator interface 134. For example, a healthcare education professional or another interested party can access how the HE content is being selected and curated in response to requests for content by multiple users. The administrator interface 134 can include options for the users to update tags/labels or other contextual data for the HE content to update/adjust how the HE content is being selected by the system 102 for presentation.

In some implementations, HE content includes live (e.g., real-time) content. Live content can include, for example, webinars, live-streamed courses, etc. The system 102 can provide, for presentation in the user interface, the live HE content options, where a user can register to participate in the live HE content, e.g., as depicted in the example UI view of FIG. 6J.

In some implementations, as described with reference to FIG. 1, the interactive healthcare education system 102 facilitates generation of reflective learning-based reflective prompts for healthcare education content. FIG. 4 is a flowchart of an example process 400 for generating reflective prompts by the interactive healthcare education system. For convenience, the process 400 will be described as being performed by a system of one or more computers, located in one or more locations, and programmed appropriately in accordance with this specification. For example, an interactive healthcare education system, e.g., the system 102 of FIG. 1, appropriately programmed, can perform the process 400.

The system receives healthcare education content 402. As described with reference to FIG. 2, the system 102 generates reflective prompts during an intake process by the system of HE content (e.g., when the system receives uploaded content from a user through the interface of the interactive platform). Additionally, or alternatively, the system 102 generates reflective prompts in response to a user selecting an HE content item for viewing.

In any case, the system obtains contextual data for the HE content 404. As described with reference to FIG. 2, system 102 extracts contextual data from the HE content and stored in a repository for enriched HE content, e.g., when the HE content subsystem 104 intakes the HE content. The contextual data can include key discussion points and learning objectives for the HE content.

In some implementations, the system generates a reflective prompt in response to a request for a content item from a user. The request from the user can be, for example, a search query or a selection of one or more keywords, e.g., as depicted in the user interfaces of FIGS. 6A, 6H, and 6I. In another example, a request can be a refreshing of a content landing page (or opening of the landing page of the application environment), e.g., as depicted in FIG. 6B. In such cases, the system extracts search terms from the initiating request 406 and obtains user profile context data 408. The search terms include terms from a user-entered search query, keywords selected to initiate the HE content request, etc.

The system provides, to a reflective prompt generation model, the contextual data for the HE content 410. In the case where the reflective prompt is generated in response to a request for HE content by the user, the system additionally provides the search terms from the initiating request and the user profile context data, e.g., based on a user's history, medical/professional interests, expertise, learning objectives (e.g., a user's stated learning goals), etc.

In some implementations, as discussed above, the reflective prompt generation model, e.g., model 144, is a large language model (LLM) that is trained using training data, e.g., training data 146, and optionally refined using supervised (e.g., regression) learning. The LLM can be trained on a dataset of human reviewed context/prompt collections, e.g., tagged user reflections and associated prompts.

The system obtains, from the reflective prompt generation model, one or more reflective prompts 412. The reflective prompts 142 each include a reflective learning-based statement or question to initiate a response from the user related to the content viewed in the healthcare education content. For example, a reflective prompt can be “How might you implement what you learned into your practice?” In another example, a reflective prompt can be “Describe how the technique presented can be beneficial over current standards of care.”

In some implementations, the one or more generated reflective prompts for an item of HE content 136 are stored in a repository of reflective prompts 142. The stored reflective prompts can be obtained by the system 102 in response to a request for the HE content for which the reflective prompts are generated. In other words, reflective prompts generated by the system 102 when the HE content is initially processed by the system can be stored and tagged for the HE content such that the system 102 can access and provide the generated reflective prompts with the HE content.

In some implementations, the one or more generated reflective prompts are provided 414 for presentation in the user interface 132 on user device 110, before, during, and/or after the presentation of the HE content. For example, a reflective prompt can be presented in an overlay or pop-up window of the application environment 116, e.g., as depicted in the example UI of FIG. 6F. Further details related to providing the HE content and one or more reflective prompts is described with reference to FIG. 5.

In some implementations, the system 102 can receive a request through the interface, e.g., user interface 132 or administrator interface 134, to generate additional reflective prompts for an HE content. For example, in a case where the generated reflective prompts are not satisfactory to the user viewing the HE content, to the content creator, or to another interested party reviewing a quality of the reflective prompts. In another example, as depicted in FIG. 8C, a user of administrator interface 134 can generate and evaluate (e.g., accept or reject) one or more reflective prompts. Optionally, the user can adjust a set of terms, keywords, contextual data, etc., used by the reflective prompt generation model to generate the reflective prompts in order to adjust an output of the model.

In some implementations, system 102 can provide a blank entry space for presentation in the user interface 132 on user device 110, before, during, and/or after the presentation of the HE content. In such cases, the user can provide a reflection in the blank entry space without directly answering a provided reflective prompt. For example, the user can provide a free-form reflective response to the HE content rather than a directed reflection in response to the reflective prompt. In some implementations, a user may select to respond as a free-form answer and/or in response to a reflective prompt. In such cases, a reflection generated as a free-form response is evaluated by the system 102 in a manner similar to the methods described with respect to a reflection provided in response to a reflective prompt, however, the system will account for the free-form nature (e.g., context free) of the response instead of including the reflective prompt as an aspect of the model-based evaluation of the reflection.

In some implementations, as described with reference to FIG. 1, the interactive healthcare education system 102 facilitates reflection analysis and HE credit classification. FIG. 5 is a flowchart of an example process 500 for processing and scoring received reflections in response to reflective prompts and classifying the received reflections as qualifying for HE credit or not qualifying for HE credit. For convenience, the process 500 will be described as being performed by a system of one or more computers, located in one or more locations, and programmed appropriately in accordance with this specification. For example, an interactive healthcare education system, e.g., the system 102 of FIG. 1, appropriately programmed, can perform the process 500.

The system provides 502 the HE content and one or more reflective prompts. The system 102 provides, for presentation in the user interface of the application environment, the HE content, e.g., as depicted in an example UI view of FIG. 6C. The UI view including the presented content can include feedback from users responsive to the HE content. Optionally, the system can include a discussion overlay or pop-up window through which users can view and interact with other users in response to the HE content and each other, e.g., as depicted in the example UI views of FIGS. 6D and 6E.

The system 102 provides, for presentation in a user interface 132 of the application environment 116 on the user device 110, the one or more reflective prompts, e.g., as depicted in the example UI view of FIG. 6F. Optionally, the system may provide multiple reflective prompts from which a user may select a reflective prompt. The system 102 may provide the reflective prompt in the same UI view as the HE content such that the reflective prompt is viewable by the user while the user is viewing the HE content.

The system 102 receives 504 a user input reflection responsive to provided reflective prompt. For example, the system can receive a reflection input into a comment section or in a dialog box presented with the reflective prompt, e.g., as depicted in the example UIs of FIGS. 6D and 6F. The user-provided reflection can include, for example, text-based content, audio-based content, video-based content, or a combination thereof.

The system 102 provides 506 the user input reflection, the corresponding reflective prompt for the reflection, and contextual data for the HE content viewed by the user to trained machine-learning models 144. At times, the system 102 can pre-process the user input reflection, e.g., to extract candidate text from the user reflection, e.g., using natural language processing, speech-to-text, or other similar techniques to yield a transcript of the user reflection in a format that can be input by the system into the models 144.

Each of the models 144 can receive a respective input including the candidate text as well as additional context, e.g., contextual data for the HE content, user profile information, search query terms used by the user to yield the HE content, etc. Models 144, as depicted in FIG. 5, include an inquisitiveness model 508, a toxicity model 510, a biomedical NRE model 512, a self-reflection model 514, and a linguistic acceptability model 516. Each of models 144 is a trained machine-learning model. Example machine-learning models that can be used for each of models 508, 510, 512, 514, and 516 include a large language model (LLM). In some instances, a convoluted neural network can be used, e.g., to parse grammar of a reflection. For example, the CNN can be used to parse grammar of a reflection and identify elements of self-reflection.

The models 144 are trained using training data 146, where the training data used to train each of the models 144 can be different for one or more of the models, e.g., where each model is trained with a different set of training data. In some implementations, models can be trained using CSV files, e.g., a first column for text of terms/phrases and a second column with a tag for the term/phrase, e.g., toxic/non-toxic etc. In some implementations, a model 144, e.g., the biomedical NRE model 512, stores the tag along with start/end offsets for the keywords/phrases to identify specific word/tag pairings in the source text.

Inquisitiveness model 508 provides, as output, a decision including a confidence score for the decision, e.g., an inquisitiveness value or check and a confidence score for the returned value or check. For example, the inquisitiveness model can output a value between 0 and 1 or a pass/fail check (e.g., pass if a threshold inquisitiveness is met, fail otherwise), and an associated confidence with the output value or check. The inquisitiveness model is trained to determine if a user is asking a question, and in the case in which a question is being asked, the type of question. For example, a question can be “can you explain what trending numbers means in terms of pathology if they are going up?”

Toxicity model 510 provides, as output, a decision including a confidence score for the decision, e.g., a toxicity value or check and a confidence score for the returned value or check. For example, the toxicity model can output a value between 0 and 1 or a pass/fail check (e.g., pass if toxicity of the response is below a threshold, fail otherwise). The toxicity model is trained to determine the extent to which the user reflection includes language identified as hateful or negative speech. For example, a user's reflection including toxic language can be “This comment is stupid.”

Biomedical NRE model 512 provides, as output, a list of possible entities, a respective position within the response text, and a classification tag for the list of possible entities, as well as a confidence score for the list of possible entities. For example, the Biomedical NRE model can output a list of possible biomedical terms/phrases, their respective positions within the response text, a classification tag for the list of biomedical terms/phrases (e.g., procedural, anatomical, surgical, etc.), and a confidence score for the list of biomedical terms/phrases (e.g., a confidence that each term/phrase is in fact biomedically related). The biomedical model 512 is trained on clinical text to annotate biological systems, lab, diagnostics, therapeutic procedures, and the like. The biomedical model is trained to determine the extent to which the user reflection includes medical entities present in the user reflection text. For example, a user reflection including biomedical terminology can be “It's better to put the tube in the small bowel instead of the stomach or gastric regions.”

Self-reflection model 514 provides, as output, a decision including a confidence score for the decision, e.g., a self-reflection value or check and a confidence score for the returned value or check. For example, the self-reflection model can output a value between 0 and 1 or a pass/fail check (e.g., pass if self-reflection of the response meets a threshold, fail otherwise). The self-reflection model is trained to determine the extent to which the user reflection includes language indicative of introspective thought. For example, a user reflection including introspective thought can be “I'm going to incorporate this into my practice!”

Linguistic acceptability model 516 provides, as output, a decision including a confidence score for the decision, e.g., a linguistic acceptability value or check and a confidence score for the returned value or check. For example, the linguistic acceptability model can output a value between 0 and 1 or a pass/fail check (e.g., pass if linguistic acceptability of the response meets a threshold, fail otherwise). The linguistic acceptability model 516 can be trained to determine the extent to which the user reflection is grammatically correct and satisfies a minimum number of words. In other words, the linguistic acceptability model can be trained to validate sentence structure and provide a rating for a level of complexity or prediction of a level of education, e.g., such that the reflection meet a (e.g., college) level reading level. For example, a user reflection not meeting a linguistic acceptability (due to being grammatically incorrect) can be “would for you to do a video on the newest clinical trials?” Additionally, the linguistic acceptability model 516 can determine if the user has engaged in plagiarism. The linguistic acceptability model can include tolerance thresholds for linguistic imperfections in lengthier sentences. For example, the model can be trained to expect that user reflections may include a nominal number of typographical errors.

The system 102 receives, from the models 508, 510, 512, 514, and 516, respective scoring criteria from the models and evaluates 518 the reflection assessment criteria. The multiple reflection assessment criteria corresponding to each of the models 508, 510, 512, 514, and 516 can be used to generate a user reflection score for the user reflection. Reflection assessment criteria for the user reflection includes multiple standards for evaluating a quality of the user reflection and can be an indicator of the user's perceived learning from the healthcare education content. In other words, a higher quality user reflection can indicate that the user has integrated the information from the HE content more effectively than a lower quality user reflection. The scoring criteria from the models can combine to produce a non-obvious assessment of the respective qualities determined by the models to determine whether or not the reflection represents a threshold scoring criteria for earning a HE credit. In other words, the respective outputs of the models, either alone or in combination, can provide insight into the value or quality of the user reflection that may not otherwise be understood, e.g., by a human evaluator. Moreover, by using trained models to evaluate the user reflections, the interface reduces the needs to have qualified human reviewers having expertise amongst many different medical and healthcare fields to evaluate the user reflections.

In some implementations, each of the models 508, 510, 512, 514, and 516 outputs a scoring value between 0 and 1. A threshold scoring value for each of the models 508, 510, 512, 514, and 516 can depend in part on the type of quality or quantity extracted by the respective model. For example, a threshold scoring value for toxicity can be a lower threshold scoring value (e.g., indicative of a low tolerance for toxic language in the user reflection), whereas a threshold scoring value for linguistic acceptability can be a higher threshold scoring value (e.g., indicative of a high expectation of linguistic complexity used by healthcare trainees and healthcare professionals).

In some implementations, the system 102 evaluates the scoring criteria by validating that, for each model 508, 510, 512, 514, and 516, a respective scoring value meets at least a respective threshold value for each of the scoring criteria. For example, for an output of the linguistic acceptability model, a threshold scoring value can be 0.7 (e.g., on a scale between 0 to 1). The system 102 can evaluate the scoring criteria on the basis of the user reflection (A) does not include toxic speech, (B) is linguistically acceptable, and (C) includes a combination of inquisitiveness, self-reflection, and relevant medical terminology.

The system 102 can evaluate the scoring values for each of the multiple reflection assessment criteria individually or as an aggregate. For example, the system 102 can evaluate each reflection assessment criterion with respect to its threshold scoring criteria. In another example, the system 102 can aggregate the scoring values for the multiple reflection assessment criteria and evaluate the aggregate scoring values with respect to a threshold aggregate scoring criterion.

In some implementations, system 102 implements a weighted scoring criteria, where scoring values from the multiple reflection assessment criteria can be weighted with respect to at least one other scoring value. For example, a poor score of inquisitiveness can be offset by a high score of the biomedical NRE terminology, based on a weighting of the respective scoring values.

In some implementations, a failing score from one or more models can disqualify the user reflection, regardless of the outcomes of the other models. For example, a reflection having a threshold toxic output (e.g., a threshold value or fail check) can result in the system disqualifying the user reflection regardless of the scoring criteria from the other models 144. In another example, an output from the linguistic acceptability model indicative of a user reflection having less than a threshold linguistic acceptability value or a fail check can disqualify the user reflection, regardless of the outcomes of the other models, e.g., having fewer than a threshold number of words or including gibberish.

In some implementations, a threshold scoring criteria from at least two of three of the inquisitiveness model, biomedical NRE model, and self-reflection model can be used by the system to classify the user reflection. The weighting of the scores of each of the three aforementioned models can be the same or a different weighting. e.g., 33% each when evaluating a cumulative score. For example, a threshold score from each of the (A) inquisitiveness model and biomedical NRE model, (B) inquisitiveness model and self-reflection model, or (C) biomedical NRE model and self-reflection models can be used to classify the user reflection as qualifying or not qualifying of an HE credit.

The system 102 classifies 520 a user reflection, based in part on the evaluation of the scoring criteria. The system 102 can classify the user reflection with the presented healthcare education content item based on whether the user reflection meets a threshold healthcare education credit criteria. In some implementations, the system 102, in response to determining that the user reflection score meets the threshold healthcare education credit criteria, classifies the user reflection as healthcare education credit qualified. In some implementations, the system 102, in response to determining that the user reflection score does not meet the threshold healthcare education credit criteria, classifies the user reflection as healthcare education credit unqualified.

The system provides 522, for presentation in the user interface, a notification including the classification of the user reflection. In the case that the system classifies the user reflection as HE credit qualified, the system 102 can provide, for presentation in the user interface, a notification in an overlay or pop-up window to notify the user that the credit is awarded, e.g., as depicted in the example UI of FIG. 6G. The system can also provide, for presentation in the user interface, an updated HE credits log in a user profile page, e.g., as depicted in the example UI of FIG. 7C. Additionally, the user profile page can include a listing of the HE content that the user has viewed and for which has earned HE credits.

In some implementations, a user of the system 102 can gain HE credits based on engagement of the user with one or more other users. For example, the system 102 can capture and analyze dialog between two or more users. In a similar manner as described above with respect to the use of the models 144 to capture the quality of the user reflection in response to the HE content, the system 102 can implement one or more models 144 to capture the quality of the user dialog and classify the dialog based on multiple reflection assessment criteria. In response to determining that the dialog meets a threshold criteria, the system 102 can classify the dialog as qualifying of HE credit.

In some implementations, the system 102 can provide, for presentation in the user interface, a HE credit report for a user and facilitate, through the user interface 132, for a user to select to send the HE credit report, e.g., to a licensing entity. The system 102 can additionally provide access to a user's HE credit report through the administrator interface 134, e.g., for access by an interested party.

In some implementations, as depicted in FIGS. 8A, 8B, the administrator interface 134 is updated to reflect the processing by the system of the user reflection in response to the HE content. The interface 134 can include, for example, user profile information, user provided reflection, and details related to the classification of the user reflection based on the threshold healthcare education credit criteria (e.g., qualified or unqualified). An administrative user can drill through each of the entries for the HE content and view additional details of the outcomes of the multiple reflection assessment criteria generated by the models 508, 510, 512, 514, and 516. For example, an administrator can view, for a selected user reflection, the outcomes of each of the multiple reflection assessment criteria, and which of the multiple reflection assessment criteria failed to meet the threshold criteria. In some implementations, an administrative user can override or modify the system classification, e.g., to grant or deny the HE credit differently than as assessed by the system 102.

In some implementations, the system 102 can assist content creation and management though the administrator interface. As depicted in the example UI view of FIG. 8C, the administrator interface can facilitate viewing/modifying of the backend content (e.g., contextual data 138) for HE content 136. For example, a user can interact with the UI to modify one or more of the title, body of text, categories (e.g., search keywords), top learning objectives, etc., for an HE content item using the administrator interface. At times, the system 102 can store and present learning objectives generated for the HE content or general learning objectives generic to the HE content, e.g., as depicted in the example UI view of FIG. 8D. A user can interact with the UI to select top learning objectives that are used to generate reflective prompts for HE content, e.g., as depicted in the example UIs of FIG. 8D, 8E, and 8F. Selecting the top learning objectives can allow the user to customize the learning objectives for a particular post. A user may select to modify the top learning objectives used to generate reflection prompts, e.g., in response to determining that one or more of the learning objectives that the system determines result in lower success rates of user reflections. For example, a user can select/deselect which of the presented learning objectives are included in the list of top learning objectives for the HE content, e.g., as depicted in the example UI views of FIGS. 8E and 8F.

In some implementations, the system 102 can provide feedback about an efficacy of one or more of the learning objectives selected as top learning objectives. The system 102 can identify learning objectives for which generated reflection prompts result in user reflections that do not qualify for HE credit. For example, if the system 102 determines that a threshold percentage of responses to reflections prompts generated in response to an HE content are failing to qualify for credit, the system 102 may suggest to the user (or automatically implement) to re-select the learning objectives used to generate user reflection prompts.

Generally, the system can collect and store data related to (but not limited to) reflection prompts and user reflections responsive to the reflection prompts to gain insight into the effectiveness/quality of the reflection prompts that are being generated (over all or for particular HE content items. For example, looking at a ‘conversion rate’ of a prompt or type of prompt into a credit can be used as a feedback loop implementable without human intervention (i.e., without an admin performing quality control) to modify the types of reflection prompts generated for a HE content item(s), e.g., by removing (deselecting) learning objectives determined to have lower success rates.

In some implementations, the system 102 can export an audit log (e.g., in CSV, JSON, etc.). Interested parties, e.g., accreditation entities, can use this log to review the user's interactions that led to granting the CME credit. The audit logs can include the date/time, duration, learning objectives, and responses to the reflective prompts that were used to award the credit.

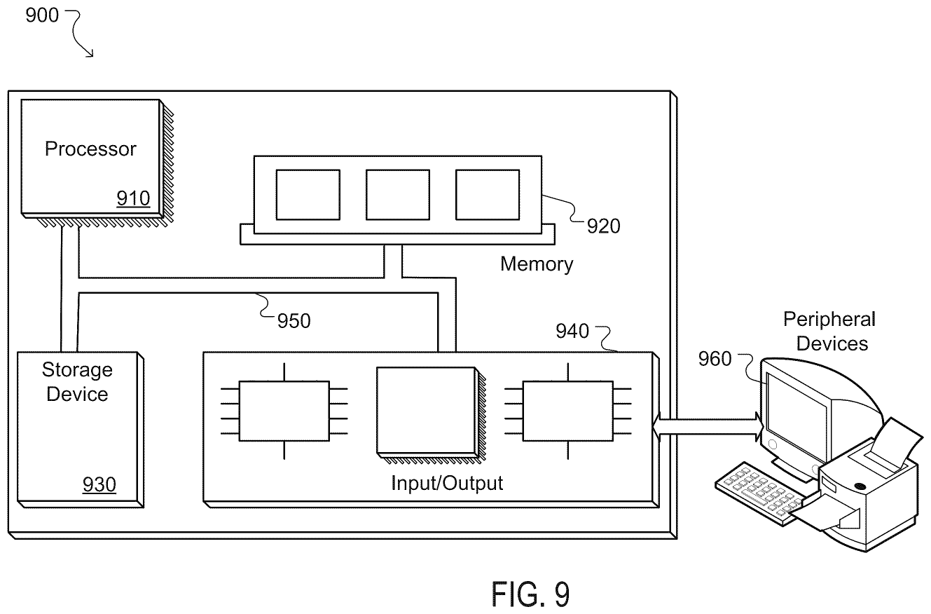

FIG. 9 is a block diagram of an example computer system 900 that can be used to perform operations described above. The system 900 includes a processor 910, a memory 920, a storage device 930, and an input/output device 940. Each of the components 910, 920, 930, and 940 can be interconnected, for example, using a system bus 950. The processor 910 is capable of processing instructions for execution within the system 900. In one implementation, the processor 910 is a single-threaded processor. In another implementation, the processor 910 is a multi-threaded processor. The processor 910 is capable of processing instructions stored in the memory 920 or on the storage device 930.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In one implementation, the storage device 930 is a computer-readable medium. In various different implementations, the storage device 930 can include, for example, a hard disk device, an optical disk device, a storage device that is shared over a network by multiple computing devices (e.g., a cloud storage device), or some other large capacity storage device.

The input/output device 940 provides input/output operations for the system 900. In one implementation, the input/output device 940 can include one or more of a network interface device, e.g., an Ethernet card, a serial communication device, e.g., and RS-232 port, and/or a wireless interface device, e.g., and 802.11 card. In another implementation, the input/output device can include driver devices configured to receive input data and send output data to peripheral devices 960, e.g., keyboard, printer and display devices. Other implementations, however, can also be used, such as mobile computing devices, mobile communication devices, set-top box television client devices, etc.

Although an example processing system has been described in FIG. 9, implementations of the subject matter and the functional operations described in this specification can be implemented in other types of digital electronic circuitry, or in computer software, firmware, or hardware, including the structures disclosed in this specification and their structural equivalents, or in combinations of one or more of them.

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively, or in addition, the carrier can be an artificially-generated propagated signal, e.g., a machine-generated electrical, optical, or electromagnetic signal, which is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random-access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. The mass storage devices can be, for example, magnetic, magneto-optical, or optical disks, or solid-state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech or tactile; and input from the user can be received in any form, including acoustic, speech, or tactile input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

This specification uses the term “configured to” in connection with systems, apparatus, and computer program components. That a system of one or more computers is configured to perform particular operations or actions means that the system has installed on its software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In this specification, the term “database” refers broadly to refer to any collection of data: the data does not need to be structured in any particular way, or structured at all, and it can be stored on storage devices in one or more locations. Thus, for example, the index database can include multiple collections of data, each of which may be organized and accessed differently.

As used in this specification, the term “engine” or “software engine” refers to a software implemented input/output system that provides an output that is different from the input. An engine can be an encoded block of functionality, such as a library, a platform, a software development kit (“SDK”), or an object. Each engine can be implemented on any appropriate type of computing device, e.g., servers, mobile phones, tablet computers, notebook computers, music players, e-book readers, laptop or desktop computers, PDAs, smart phones, or other stationary or portable devices, which includes one or more processors and computer readable media. Additionally, two or more of the engines may be implemented on the same computing device, or on different computing devices.

In situations in which the systems discussed here collect personal information about users, or may make use of personal information, the users may be provided with an opportunity to control whether applications or features collect user information (e.g., information about a user's social network, social actions or activities, profession, a user's preferences, or a user's current location), or to control whether and/or how to receive content that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially be claimed as such, one or more features from a claimed combination can in some cases be excised from the combination, and the claim may be directed to a sub combination or variation of a sub combination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method for administering managed delivery of customized content related to healthcare education (HE) on a user device, the method comprising:
selecting a plurality of reflection assessment criteria for evaluating user reflections responsive to a user engaging with the HE content items, the selected reflective assessment criteria to characterize the user reflections as indicative of a user engaging in reflective learning responsive to HE content items;
generating a plurality of training data sets, each training data set constructed to train a corresponding large language model (LLM) that evaluates the user reflections responsive to HE content items based on a corresponding selected reflection assessment criteria, wherein the generating of the plurality of training data sets comprises:
extracting, from a plurality of HE content items, terms and phrases labeled in accordance with the selected reflection assessment criteria;
generating, from the plurality of HE content items, contextual data including terms and phrases defining sets of learning objectives pertaining to the plurality of HE content items; and
generating the corresponding training data sets by storing, in respective data structures, (i) the terms and phrases and (ii) tags for the terms and phrases labeled in accordance with the corresponding selected reflection assessment criteria;
training each of a plurality of LLMs using the corresponding training data set of the plurality of training data sets
generating, using the contextual data for the HE content item and by a corresponding LLM of the plurality of LLMS, corresponding reflection prompts for the plurality of HE content items, each reflection prompt configured to engage a user in reflective learning targeting a set of generated learning objectives for the HE content item;
providing, for presentation on a user interface and in response to requests for health care education content, a selected HE content item and a corresponding reflection prompt;
receiving, through the user interface, a user reflection responsive to the reflection prompt;
evaluating the user reflection as indicative of the user engaging in reflective learning responsive to the selected HE content item, comprising:
providing, as input to the plurality of LLMs, the user reflection, the corresponding reflection prompt, and the corresponding contextual data for the selected HE content item; and
generating a cumulative score value comprising a weighted combination of respective scoring values for the user reflection output by the plurality of LLMs according to a weighted scoring criteria defining a relative weight of each of the selected reflective assessment criteria with respect to each other; and
providing, through the user interface and based on the cumulative score value, a notification indicative of the user's engagement in reflective learning for the selected HE content item.

2. The method of claim 1, wherein the reflection assessment criteria correspond to (A) a toxicity, (B) a linguistic complexity, (C) a medical terminology, (D) an inquisitiveness, and (E) a self-reflection of the user reflection.

3. The method of claim 1, wherein generating contextual data for the HE content item comprises:
generating, from text-based transcription of the HE content item, a set of key discussion points for the HE content item; and
identifying, for the set of key discussion points, one or more learning objectives for the HE content item.

4. (canceled)

5. (canceled)

6. (canceled)

7. (canceled)

8. The method of claim 1, wherein a selected reflection assessment criteria is weighted differently than at least one other second reflection assessment criteria.

9. The method of claim 1, wherein the notification indicative of the user's engagement in reflective learning for the selected HE content comprises:
determining a continuing healthcare education credit value for the user reflection; and
providing the continuing healthcare education credit value for recordation by a medical licensing entity.

10. (canceled)

11. (canceled)

12. One or more non-transitory computer storage media encoded with computer program instructions that when executed by one or more computers cause the one or more computers to perform operations of administering managed delivery of customized content related to healthcare education (HE) on a user device comprising:
selecting a plurality of reflection assessment criteria for evaluating user reflections responsive to a user engaging with the HE content items, the selected reflective assessment criteria to characterize the user reflections as indicative of a user engaging in reflective learning responsive to HE content items;
generating a plurality of training data sets, each training data set constructed to train a corresponding large language model (LLM) that evaluates the user reflections responsive to HE content items based on a corresponding selected reflection assessment criteria, wherein the generating of the plurality of training data sets comprises:
extracting, from a plurality of HE content items, terms and phrases labeled in accordance with the selected reflection assessment criteria;
generating, from the plurality of HE content items, contextual data including terms and phrases defining sets of learning objectives pertaining to the plurality of HE content items; and
generating the corresponding training data sets by storing, in respective data structures, (i) the terms and phrases and (ii) tags for the terms and phrases labeled in accordance with the corresponding selected reflection assessment criteria;
training each of a plurality of LLMs using the corresponding training data set of the plurality of training data sets;
generating, using the contextual data for the HE content item and by a corresponding LLM of the plurality of LLMS, corresponding reflection prompts for the plurality of HE content items, each reflection prompt configured to engage a user in reflective learning targeting a set of generated learning objectives for the HE content item;
providing, for presentation on a user interface and in response to requests for health care education content, a selected HE content item and a corresponding reflection prompt;
receiving, through the user interface, a user reflection responsive to the reflection prompt;
evaluating the user reflection as indicative of the user engaging in reflective learning responsive to the selected HE content item, comprising:
providing, as input to the plurality of LLMs, the user reflection, the corresponding reflection prompt, and the corresponding contextual data for the selected HE content item; and
generating a cumulative score value comprising a weighted combination of respective scoring values for the user reflection output by the plurality of LLMs according to a weighted scoring criteria defining a relative weight of each of the selected reflective assessment criteria with respect to each other; and
providing, through the user interface and based on the cumulative score value, a notification indicative of the user's engagement in reflective learning for the selected HE content item.

13. The one or more non-transitory computer storage media of claim 12, wherein the reflection assessment criteria correspond to (A) a toxicity, (B) a linguistic complexity, (C) a medical terminology, (D) an inquisitiveness, and (E) a self-reflection of the user reflection.

14. The one or more non-transitory computer storage media of claim 12, wherein generating contextual data for the HE content item comprises:
generating, from text-based transcription of the HE content item, a set of key discussion points for the HE content item; and
identifying, for the set of key discussion points, one or more learning objectives for the HE content item.

15. (canceled)

16. (canceled)

17. (canceled)

18. (canceled)

19. (canceled)

20. A system comprising:
one or more computers and one or more storage devices on which are stored instructions that are operable, when executed by the one or more computers, to cause the one or more computers to perform operations comprising:
selecting a plurality of reflection assessment criteria for evaluating user reflections responsive to a user engaging with the HE content items, the selected reflective assessment criteria to characterize the user reflections as indicative of a user engaging in reflective learning responsive to HE content items;
generating a plurality of training data sets, each training data set constructed to train a corresponding large language model (LLM) that evaluates the user reflections responsive to HE content items based on a corresponding selected reflection assessment criteria, wherein the generating of the plurality of training data sets comprises:
extracting, from a plurality of HE content items, terms and phrases labeled in accordance with the selected reflection assessment criteria;
generating, from the plurality of HE content items, contextual data including terms and phrases defining sets of learning objectives pertaining to the plurality of HE content items; and
generating the corresponding training data sets by storing, in respective data structures, (i) the terms and phrases and (ii) tags for the terms and phrases labeled in accordance with the corresponding selected reflection assessment criteria;
training each of a plurality of LLMs using the corresponding training data set of the plurality of training data sets;
generating, using the contextual data for the HE content item and by a corresponding LLM of the plurality of LLMS, corresponding reflection prompts for the plurality of HE content items, each reflection prompt configured to engage a user in reflective learning targeting a set of generated learning objectives for the HE content item;
providing, for presentation on a user interface and in response to requests for health care education content, a selected HE content item and a corresponding reflection prompt;
receiving, through the user interface, a user reflection responsive to the reflection prompt;
evaluating the user reflection as indicative of the user engaging in reflective learning responsive to the selected HE content item, comprising:
providing, as input to the plurality of LLMs, the user reflection, the corresponding reflection prompt, and the corresponding contextual data for the selected HE content item; and
generating a cumulative score value comprising a weighted combination of respective scoring values for the user reflection output by the plurality of LLMs according to a weighted scoring criteria defining a relative weight of each of the selected reflective assessment criteria with respect to each other; and
providing, through the user interface and based on the cumulative score value, a notification indicative of the user's engagement in reflective learning for the selected HE content item.

21. (canceled)

22. (canceled)

23. (canceled)

24. (canceled)

25. The method of claim 1, wherein extracting terms and phrases comprises extracting, from enriched healthcare content items, associations between discussion points and learning objectives for the HE content items.

26. (canceled)

27. The method of claim 1, wherein providing the selected HE content item and the corresponding reflection prompt comprises providing a curated subset of healthcare education content items using historical user behavior for the user, comprising:
determining, from historical user behavior for the user, one or more of conversation topics for the user, topics of previously viewed healthcare content items, and respective times of interactions with the previously viewed healthcare content items;
selecting, from the plurality of HE content items and using the historical user behavior of the user, the curated subset of healthcare education content items; and
providing, the curated subset of healthcare education content items for presentation in the user interface.

28. The method of claim 27, wherein providing the curated subset of healthcare education content items further comprises:
providing, for presentation in the user interface, selectable controls for filtering the curated subset of healthcare education content items, wherein the selectable controls includes filtering the curated subset of healthcare education content items by duration of content, media type, and expected length of interaction;
receiving, through the user interface, an interaction with one or more of the selectable controls; and
providing, in response to the interaction and for presentation in the user interface, a proper subset of the curated subset of healthcare education content items.

29. The method of claim 1, wherein providing the one or more reflective prompts comprises:
providing, for presentation in the user interface, at least one of the one or more reflective prompts in a same view of the user interface as the HE content item such that the prompt is viewable by the user while the user is viewing the selected HE content item.
